(19) United States
(12) Patent Application Publication
(10) Pub. No.: US 2014/0221815 A1
(43) Pub. Date: Aug. 7, 2014

(54) SUPPORT UNIT FOR POSITIONING A PATIENT

(71) Applicant: SIEMENS AKTIENGESELLSCHAFT, Munich (DE)

(72) Inventors: Bassim AKLAN, Erlangen (DE); Annemarie HAUSOTTE, Erlangen (DE); René KARTMANN, Nuremberg (DE); Ralf LADEBECK, Erlangen (DE); Daniel PAULUS, Nuremberg (DE); Harald H. QUICK, Erlangen (DE)

(73) Assignee: SIEMENS AKTIENGESELLSCHAFT, Munich (DE)

(21) Appl. No.: 14/102,910

(22) Filed: Dec. 11, 2013

(30) Foreign Application Priority Data
Feb. 1, 2013 (DE) 102013201700.6

(51) Int. Cl. A61B 5/055; G01R 33/48

(52) U.S. Cl. CPC A61B 5/0555; G01R 33/481
USPC 600/411; 600/415

(57) ABSTRACT

A support unit is disclosed for positioning a patient within a medical imaging apparatus with at least one magnetic resonance device; along with a patient positioning apparatus and a medical imaging apparatus including such a support unit. In an embodiment, the support unit includes at least one bearing element for movable positioning relative to a patient positioning apparatus and a coil unit for receiving magnetic resonance raw data. The coil unit includes a flat supporting surface for positioning the patient.

### PRIORITY STATEMENT

The present application hereby claims priority under 35 U.S.C. §119 to German patent application number DE 102013201700.6 filed Feb. 1, 2013, the entire contents of which are hereby incorporated herein by reference.

### FIELD

At least one embodiment of the invention generally relates to a support unit for positioning a patient within a medical imaging apparatus with at least one magnetic resonance device, a patient positioning apparatus, and a medical imaging apparatus having such a support unit.

### BACKGROUND

Conventional support units are used in the field of medical technology, in particular in the field of imaging methods. It is frequently necessary for the patient or the body region under examination, for example the upper body, to be positioned in as stable a manner as possible in a specific position. In practice tabletop platforms are frequently used to position or fix for example the upper body in a desired manner.

### SUMMARY

At least one embodiment of the invention is directed to to a support unit for positioning a patient within a medical imaging apparatus with at least one magnetic resonance device such that during the application of the medical-technical method in use, a position of a patient or of specific body regions is ensured that is as variable, correct and secure as possible.

A support unit, a patient positioning apparatus and a medical imaging apparatus are disclosed. Advantageous embodiments of the invention are set out in the dependent claims.

In an embodiment, a support unit is provided for positioning a patient within a medical imaging apparatus with at least one magnetic resonance device. The support unit includes at least one bearing element for movable positioning relative to a patient positioning apparatus and a coil unit that in turn comprises a flat supporting surface for positioning the patient. The coil unit here serves as a radio-frequency antenna unit for receiving magnetic resonance raw data. A roller bearing, a journal bearing or another element embodied for movable positioning, for example, can be deployed as a bearing element.

A medical imaging apparatus that comprises at least one magnetic resonance device is also disclosed within the scope of an embodiment of the invention. According to the embodiment variant this includes a support unit and/or a patient positioning apparatus and/or an encoding element and/or an identification unit. Owing to the optimized distance between the coil unit and the patient, furthermore, the signal of the medical imaging apparatus is less strongly attenuated than would be the case if a conventional unit and a tabletop platform were deployed.

### BRIEF DESCRIPTION OF THE DRAWINGS

The invention is described and explained in more detail below with reference to the exemplary embodiments illustrated in the figures, in which:

FIG. 1 shows a schematic representation of a conventional coil unit with a tabletop platform,

FIG. 2 shows a schematic representation of a support unit according to an embodiment of the invention, and

FIG. 3 shows a magnetic resonance device with a patient positioning apparatus according to an embodiment of the invention and a support unit.

### DETAILED DESCRIPTION OF THE EXAMPLE EMBODIMENTS

The present invention will be further described in detail in conjunction with the accompanying drawings and embodiments. It should be understood that the particular embodiments described herein are only used to illustrate the present invention but not to limit the present invention.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

An embodiment of the invention is based on the knowledge that, through suitable design engineering of a support unit with at least one bearing unit and one coil unit, the distance between the coil unit and a patient can be optimized such that the signal-to-noise ratio can be increased and thereby the image quality of the recording with the magnetic resonance device or medical imaging apparatus with at least one magnetic resonance device can be improved. Furthermore additional cushions or towels intended to level out any unevenness of a conventional coil unit can be dispensed with. Thus the support unit also facilitates or at least supports faster diagnosis and a smoother workflow.

In an embodiment, a support unit is provided for positioning a patient within a medical imaging apparatus with at least one magnetic resonance device. The support unit includes at least one bearing element for movable positioning relative to a patient positioning apparatus and a coil unit that in turn comprises a flat supporting surface for positioning the patient. The coil unit here serves as a radio-frequency antenna unit for receiving magnetic resonance raw data. A roller bearing, a journal bearing or another element embodied for movable positioning, for example, can be deployed as a bearing element.

The medical imaging apparatus can be formed by a magnetic resonance device, but also e.g. by a combined magnetic resonance/positron emission tomography device, a magnetic resonance device with linear accelerator, a magnetic resonance device with cobalt radiotherapy, or other hybrid systems.

In a particularly suitable application the coil unit includes a housing that comprises the supporting surface. Thus in a simple manner a complete system is realized that incorporates traditionally separate components in a combination. A complete system of this type not only saves space but is furthermore also economical to implement.

A further embodiment variant provides for the bearing element to be arranged on the housing of the coil unit. This ensures movable positioning of a patient within a magnetic resonance device or a combined magnetic resonance/positron emission tomography device.

An inventive embodiment includes the support unit being provided at least with one encoding element. By this, the support unit can be identified automatically by an identification unit of a magnetic resonance device or of a medical imaging apparatus with at least one magnetic resonance device. In the case of identification by a magnetic resonance device the encoding can be effected via a plug of an integral coil of the magnetic resonance device. In the case of identification by a combined magnetic resonance/positron emission tomography device, the automatic generation of an attenuation chart for the magnetic resonance/positron emission tomography device can be triggered furthermore, and account can accordingly be taken of the individual hardware components in the support unit used, such as e.g. masks, padding or other positioning aids.

A further inventive embodiment provides for the encoding element to be arranged on the coil unit. In this way the encoding element cannot be covered by a patient's body.

In an advantageous embodiment variant the encoding element is provided with an RFID transponder. By reading out an identifying code of the RFID transponder, the support unit used can also be recognized and identified.

A further advantageous embodiment variant includes an identification unit with which the aforementioned identifying code of the RFID transponder can be read out. Through this direct capture and thus direct registration of the support unit used, further workflows, in which the type and properties of the support unit are of importance, can also be simplified.

A medical imaging apparatus that comprises at least one magnetic resonance device is also disclosed within the scope of an embodiment of the invention. According to the embodiment variant this includes a support unit and/or a patient positioning apparatus and/or an encoding element and/or an identification unit with the advantages already referred to above. Owing to the optimized distance between the coil unit and the patient, furthermore, the signal of the medical imaging apparatus is less strongly attenuated than would be the case if a conventional unit and a tabletop platform were deployed.

In a particularly advantageous embodiment variant, the identification unit of the medical imaging apparatus is embodied for the provision of automatic attenuation correction for the recording of positron emission tomography image data sets as a function of the support unit used. This results in an additional time saving as the steps required to obtain an instruction for calculating an attenuation correction specific to the support unit are eliminated.

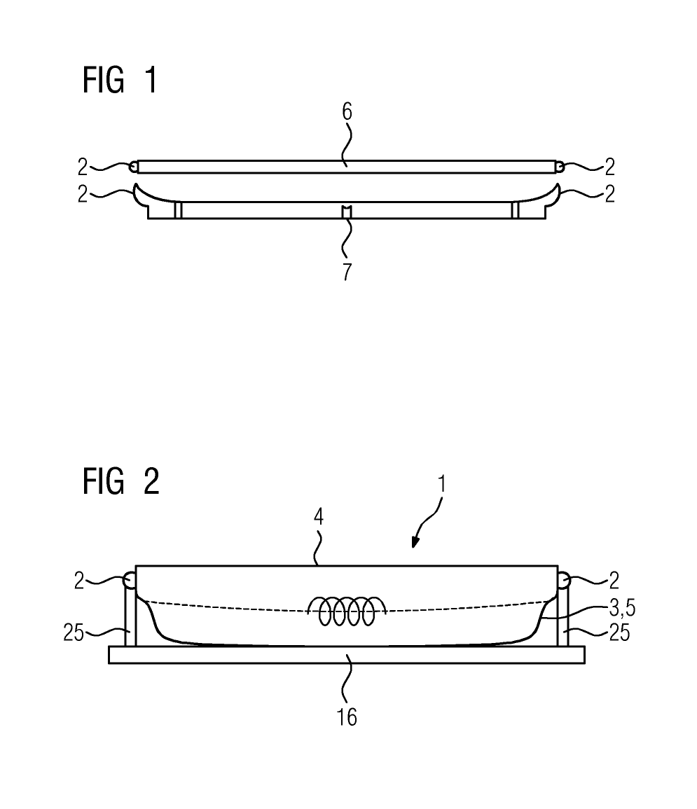

FIG. 1 shows a schematic representation of a conventional coil unit 7 with a tabletop platform 6. In this case a distance remains between the conventional coil unit 7 and the outside of the tabletop platform 6. Here the conventional coil unit 7 and the tabletop platform 6 are each provided with two bearing elements 2.

FIG. 2 shows a schematic representation of a support unit 1 according to an embodiment of the invention. The support unit 1 has two bearing elements 2 for movable positioning relative to a patient positioning apparatus 16 and a coil unit 3 for receiving magnetic resonance raw data. The coil unit 3 comprises a flat supporting surface 4 for the patient 15. The coil unit 3 furthermore has a housing 5 that comprises the supporting surface 4. The figure shows a section through the support unit 1 perpendicularly with respect to its longitudinal axis. In this view the bearing elements 2 are arranged on the side of the support unit 1.

The bearing elements 2 are additionally located on the housing 5 of the coil unit 3. The support unit 1 serves to position a patient 15 within a magnetic resonance device 10 or a combined magnetic resonance/positron emission tomography device. Movable positioning relative to a patient positioning apparatus 16 is achieved by way of the bearing elements 2. In this case the bearing elements 2 can be arranged on the side of the support unit 1, as shown in FIG. 2, but they can also be arranged on the underside of the support unit 1 for instance in the form of a guide rail. A roller bearing, a journal bearing or another element embodied for movable positioning, for example, can also be deployed as a bearing element. In this case the patient positioning apparatus 16 can be embodied for example in the form of a table on which are arranged rails into which the bearing elements 2 can in turn be inserted.

Through such design engineering the distance between the coil unit 3 and a patient 15 can be optimized such that the signal-to-noise ratio is increased and thereby the image quality of the recording with the magnetic resonance device 10 or combined magnetic resonance/positron emission tomography device is improved.

The support unit 1 can additionally be provided with an encoding element 8 that facilitates identification by a magnetic resonance device 10 or a combined magnetic resonance/positron emission tomography device. The encoding element 8 can be provided for example with an RFID transponder. By reading out the identifying code of the RFID transponder, the support unit 1 used can also be recognized and identified.

In this way an automatic attenuation correction can also be implemented for a combined magnetic resonance/positron emission tomography device. The identification unit 9 of the combined magnetic resonance/positron emission tomography device can read out the identifying code of the RFID transponder, identify the support unit 1 used, and thus with the aid of the hardware components present in the support unit 1 used generate an attenuation chart for automatic attenuation correction.

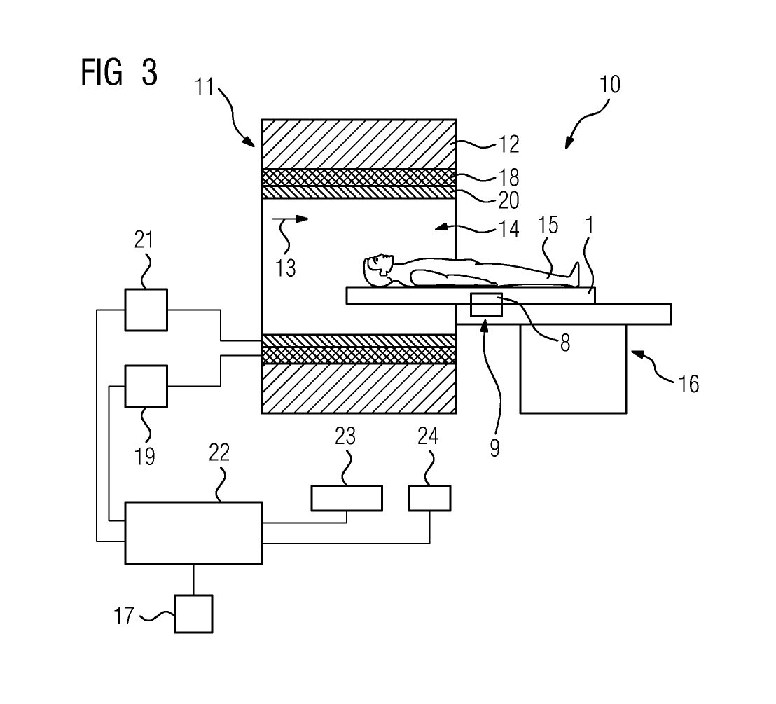

FIG. 3 shows a medical imaging apparatus 10 with a patient positioning apparatus 16 according to the invention and a support unit 1. The medical imaging apparatus 10 is formed here by a magnetic resonance device, but can also be formed e.g. by a combined magnetic resonance/positron emission tomography device, a magnetic resonance device with linear accelerator, a magnetic resonance device with cobalt radiotherapy, or other hybrid systems.

The magnetic resonance device 10 comprises a detector unit formed by a magnet unit 11 having a main magnet 12 for generating a strong and in particular constant main magnetic field 13. The magnetic resonance device 10 also has a cylinder-shaped patient examination area 14 for accommodating a patient 15, the patient examination area 14 being enclosed by the magnet unit 11 in a circumferential direction. The patient 15 can be introduced into the patient examination area 14 via a patient positioning apparatus 16 of the magnetic resonance device 10. For this purpose the patient positioning apparatus 16 has a support unit 1 that is movably arranged within the magnetic resonance device 10, in particular within the patient receiving area 15.

The magnet unit 11 additionally has a gradient coil unit 18 for generating magnetic field gradients which is used for spatial encoding during an imaging session. The gradient coil unit 18 is controlled by means of a gradient control unit 19. The magnet unit 11 also has a radio-frequency antenna unit 20 and a radio-frequency antenna control unit 21 for stimulating a polarization which becomes established in the main magnetic field 13 generated by the main magnet 12. The radio-frequency antenna unit 20 is controlled by the radio-frequency antenna control unit 21 and radiates radio-frequency magnetic resonance sequences into an examination space which is formed substantially by the patient examination area 14.

For the purpose of controlling the main magnet 12, the gradient control unit 19 and the radio-frequency antenna control unit 21, the magnetic resonance device 10 has a control unit 22 formed by a computing unit (including, e.g., a microprocessor or computer). The control unit 22 is used for central control of the magnetic resonance device 10, such as performing a predetermined imaging gradient echo sequence for example. Control information such as imaging parameters, for example, as well as reconstructed magnetic resonance images can be displayed on a display unit 23, for example on at least one monitor, of the magnetic resonance device 10 for viewing by an operator. Furthermore, the magnetic resonance device 10 has an input unit 24 by means of which information and/or parameters can be entered by an operator during a measurement procedure. Evaluation and/or processing of the magnetic resonance measurement data obtained are performed by way of a data evaluation unit 17.

The magnetic resonance device 10 shown can obviously comprise further components that magnetic resonance devices 10 typically include. Furthermore, the general mode of operation of a magnetic resonance device 10 is known to the person skilled in the art, so a detailed description of the general components will be dispensed with.

Although the invention has been illustrated and described in greater detail on the basis of the preferred example embodiments, the invention is nevertheless not limited by the disclosed examples and other variations can be derived herefrom by the person skilled in the art without departing from the scope of protection of the invention.

In summary, at least one embodiment of the invention relates to a support unit for positioning a patient within a medical imaging apparatus with at least one magnetic resonance device, a patient positioning apparatus, and a medical imaging apparatus having such a support unit. In this case the support unit has at least one bearing element for movable positioning relative to a patient positioning apparatus and a coil unit for receiving magnetic resonance raw data. The coil unit comprises a flat supporting surface for positioning the patient.

What is claimed is:

1. A support unit for positioning a patient within a medical imaging apparatus with at least one magnetic resonance device, the support unit comprising:
at least one bearing element, configured for movable positioning relative to a patient positioning apparatus; and
a coil unit, configured to receive magnetic resonance raw data, the coil unit including a flat supporting surface for positioning the patient.

2. The support unit of claim 1, wherein the coil unit includes a housing that comprises the supporting surface.

3. The support unit of claim 2, wherein the at least one bearing element is arranged on the housing of the coil unit.

4. The support unit of claim 1, wherein the support unit includes at least one encoding element for encoding the support unit.

5. The support unit of claim 4, wherein the encoding element is arranged on the coil unit.

6. The support unit of claim 4, wherein the encoding element is provided with an RFID transponder.

7. A patient positioning apparatus comprising the support unit of claim 1.

8. The patient positioning apparatus of claim 7, further comprising an identification unit for identification of the encoded support unit.

9. A medical imaging apparatus comprising:
at least one magnetic resonance device; and
the support unit for positioning a patient, of claim 1.

10. The medical imaging apparatus of claim 9, further comprising an encoding element and an identification unit.

11. The medical imaging apparatus of claim 10, wherein the identification unit is embodied for the provision of automatic attenuation correction for the recording of positron emission tomography image data sets as a function of the support unit used.

12. The support unit of claim 2, wherein the support unit includes at least one encoding element for encoding the support unit.

13. The support unit of claim 12, wherein the encoding element is arranged on the coil unit.

14. The support unit of claim 5, wherein the encoding element is provided with an RFID transponder.

15. The support unit of claim 12, wherein the encoding element is provided with an RFID transponder.

16. The support unit of claim 13, wherein the encoding element is provided with an RFID transponder.

17. A medical imaging apparatus comprising:
at least one magnetic resonance device; and
the patient positioning apparatus of claim 7.

18. The medical imaging apparatus of claim 17, further comprising an encoding element and an identification unit.

19. The medical imaging apparatus of claim 18, wherein the identification unit is embodied for the provision of automatic attenuation correction for the recording of positron emission tomography image data sets as a function of the support unit used.
